(19) United States
(12) Patent Application Publication
(10) Pub. No.: US 2021/0393976 A1
(43) Pub. Date: Dec. 23, 2021

(54) PHOTOTHERAPY DEVICE

(71) Applicant: ARBOR GRACE, INC., Detroit, MI (US)

(72) Inventors: Patrick Kenneth Powell, Detroit, MI (US); Marissa Gilbert, Harrison Township, MI (US)

(21) Appl. No.: 17/397,074

(22) Filed: Aug. 9, 2021

Related U.S. Application Data
(63) Continuation of application No. PCT/US2020/027512, filed on Apr. 9, 2020.
(60) Provisional application No. 62/831,334, filed on Apr. 9, 2019.
(60) Provisional application No. 62/958,382, filed on Jan. 8, 2020.
(60) Provisional application No. 63/092,001, filed on Oct. 15, 2020.
(60) Provisional application No. 63/064,160, filed on Aug. 11, 2020.

(51) Int. Cl. A61N 5/06; H05K 1/14; H05K 1/02

(52) U.S. Cl. CPC A61N 5/0621; H05K 1/14; H05K 1/028; H05K 2201/10106; A61N 2005/0663; A61N 2005/0626; A61N 2005/0637; A61N 2005/0633; A61N 2005/0652

(57) ABSTRACT

A phototherapy device includes at least one flexible sheet that has first and second electrical conduction layers, an electrical insulation layer between the first and second electrical conduction layers, and light emitting diodes (LEDs) disposed along the electrical insulation layer between the first and second electrical conduction layers. The first electrical conduction layer includes a conductive polymer. The electrical conduction layers and the LEDs are part of an electrical circuit in which the LEDs illuminate responsive to an electrical input. There is at least one meter to measure an electrical property of the electrical circuit. A control module is in communication with the meter and configured to adjust the electrical input responsive to the electrical property of the electrical circuit.

### BACKGROUND

Photo-treatment involves the emission of light or other radiation onto a subject. Such treatment is often used for medical purposes as a “phototherapy.” One example of phototherapy involves using light to reduce bilirubin in neonates. Light, most typically light in the visible blue portion of the spectrum, is directed at the neonate. The light absorbs through the neonate's skin and causes a photo-reaction that chemically breaks down bilirubin. Most often, a suspended light shines directly onto the neonate to provide phototherapy. More recently, however, fiber-optic blankets have also been developed.

### SUMMARY

A phototherapy device according to an example of the present disclosure includes at least one flexible sheet that has first and second electrical conduction layers. The first electrical conduction layer includes a conductive polymer, an electrical insulation layer between the first and second electrical conduction layers, and light emitting diodes (LEDs) disposed along the electrical insulation layer between the first and second electrical conduction layers. The first and second electrical conduction layers and the LEDs are part of an electrical circuit in which the LEDs illuminate responsive to an electrical input. There is at least one meter to measure an electrical property of the electrical circuit. A control module is in communication with the at least one meter and is configured to adjust the electrical input responsive to the electrical property of the electrical circuit.

A phototherapy device according to an example of the present disclosure includes a reconfigurable group of components that have first and second flexible sheets each having light emitting diodes (LEDs), a first control module, a network of electrical connection, and a wearable that is configured to be attached on anatomy of a human body. The wearable includes a second control module, a battery, and body electrical connection. The reconfigurable group of components are operable in the following configurations: a first configuration in which the first and second flexible sheets are connected via the network of electrical connections to the first control module, and the first control module is configured to adjust electrical inputs to the first and second flexible sheets, and a second configuration in which the first flexible sheet is disconnected from the network of electrical connections and first control module and is connected to the body electrical connection, and the second control module is configured to adjust electrical input to the first flexible sheet.

### BRIEF DESCRIPTION OF THE DRAWINGS

The various features and advantages of the present disclosure will become apparent to those skilled in the art from the following detailed description. The drawings that accompany the detailed description can be briefly described as follows.

FIG. 1A illustrates an example phototherapy device in a closed position.

FIG. 1B illustrates a sectioned view of the phototherapy device.

FIG. 1C illustrates a view of a portion of the phototherapy device that has a base panel.

FIG. 1D illustrates the phototherapy device in an open position.

FIG. 2 illustrates a flexible sheet with light emitting diodes.

FIG. 3 illustrates a flow diagram for controlling the phototherapy device.

FIG. 4A illustrates a reconfiguration in which a flexible sheet is used with a wearable.

FIG. 4B illustrates the wearable from the back.

### DETAILED DESCRIPTION

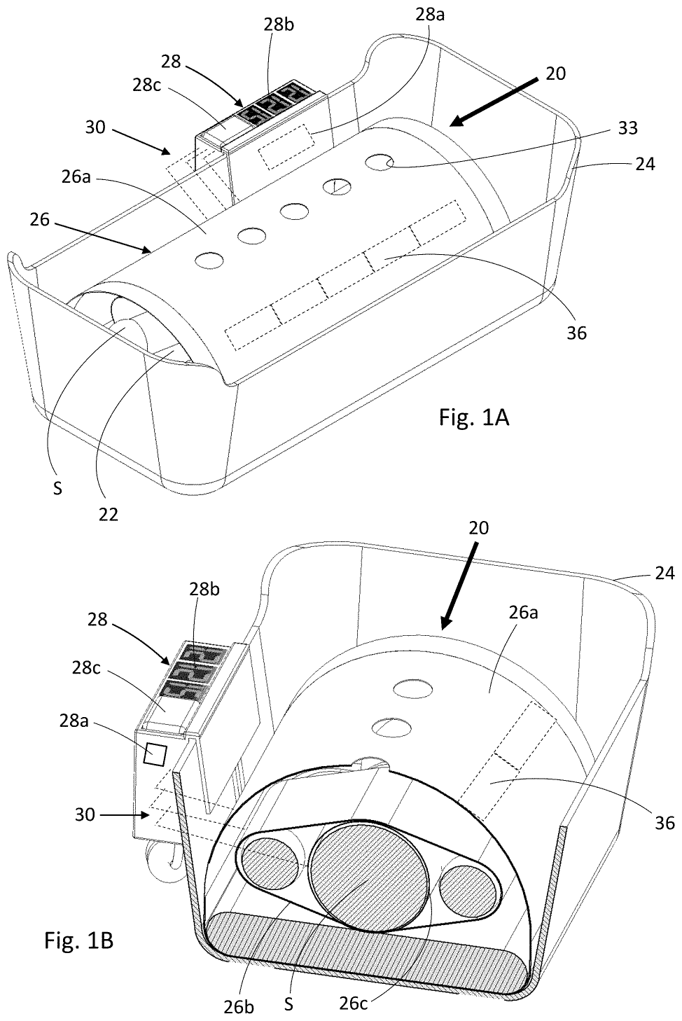

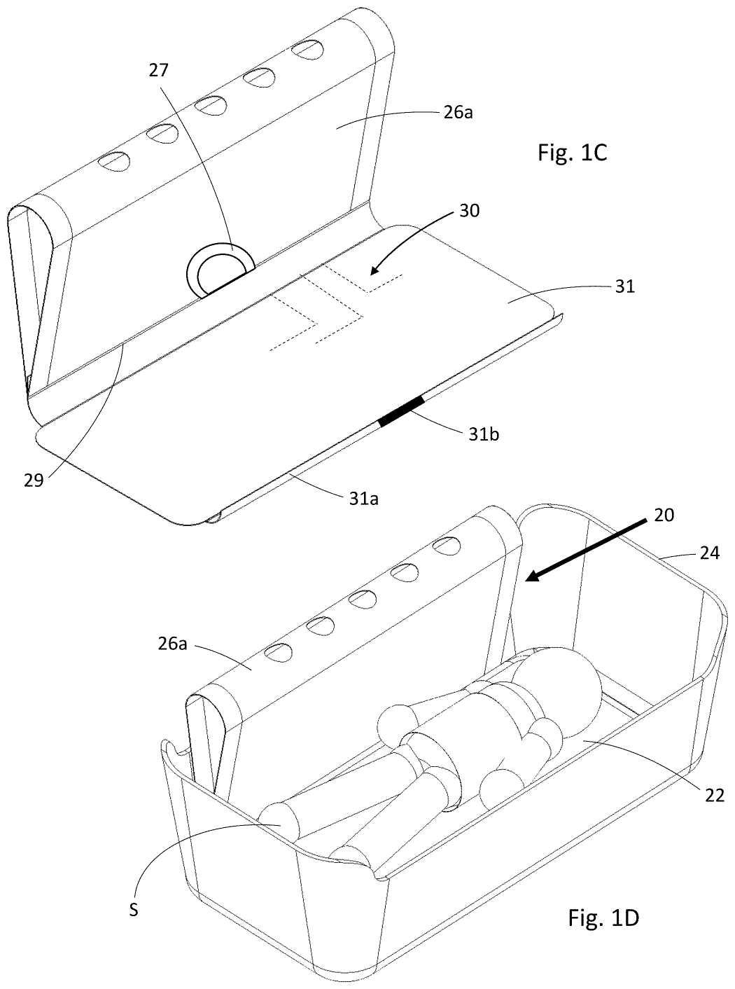

FIG. 1A schematically illustrates an example implementation of a phototherapy device 20, and FIG. 1B illustrates a sectioned view of the phototherapy device 20. As shown, the phototherapy device 20 is implemented in conjunction with a mattress 22 and a bassinet 24. It is to be understood, however, that the phototherapy device 20 could alternatively be used independently of the mattress 22 and basinet 24, with only the mattress 22, or with only the bassinet 24.

The phototherapy device 20 generally includes at least one flexible sheet 26 and a controller 28. The controller 28 generally controls operation of the flexible sheets 26 to emit light onto the subject (S). As will be discussed in further detail below, each flexible sheet 26 includes an array of light emitting diodes 36 (portion shown) for emitting light onto a treatment subject (S), such as a neonate.

In the example shown, the phototherapy device 20 includes three flexible sheets, indicated at 26a/26b/26c. As will be appreciated, modified examples of the phototherapy device 20 can include a single flexible sheet 26, two flexible sheets 26, or more than three flexible sheets 26. The flexible sheet or sheets 26 are connected with the controller 28 via a network of electrical connections 30. Although shown schematically in FIGS. 1A/1B, the electrical connections 30 may be routed under the mattress 22, or on or in a frame or base that is provided with the flexible sheet or sheets 26. For instance, FIG. 1C illustrates a portion of the phototherapy device 20 in which the flexible sheet 26a is affixed with a base panel 31, and at least a portion of the network of electrical connections 30 is provided through the base panel 31. In this regard, the electrical connections 30 may include connectors or the like for plugging in the flexible sheets 26 for operation with the controller 28. The electrical connections 30 may include at least one wireless power transfer connection between the flexible sheets 26 for operation with the controller 28. The base panel 31 may also incorporate other functional features, such as a closure 31a for attaching the flexible sheet 26a and/or a switch 31b (e.g., a magnetic reed switch) for automatic shut-off of all the flexible sheets 26 when the flexible sheet 26a is opened. For example, the closure includes, but is not limited to, a magnetic closure, hook and loop fastener, or snaps.

The flexible sheets 26a/26b/26c are functionally adapted for particular purposes, such as phototherapy for neonate jaundice. For instance, in the example shown, the flexible sheet 26a is relatively large in size and contains a high number of LEDs 36 (by number and/or lit surface area) in comparison to the other flexible sheets 26 in order to provide substantially whole body illumination. The flexible sheet 26a may also be provided with one or more windows 33 to permit viewing of the subject (S) and/or for air circulation.

The flexible sheet 26a is moveable between a closed position (FIG. 1A) and a retracted, open position, which is shown in FIG. 1D. In the closed position, the flexible sheet 26a has a curved shape, such as a half-cylindrical shape, that arcs over the subject (S). The flexible sheet 26a may be attached to the base panel 31 using the closure 31a to maintain the flexible sheet 26a in the closed position. In the retracted position, the flexible sheet 26a is folded back on itself in a loop so as to provide access to the subject (S). In this regard, the flexible sheet 26a may include a handle 27 to facilitate opening and closing and a cushioned edge 29, such as a foam lip, in case the edge of the flexible sheet 26a contacts the subject during opening and closing. For example, a method of treating jaundice using the phototherapy device 20 may include moving the flexible sheet 26a to the closed position such that it is in proximity of a jaundiced neonate subject (S) underneath and causes the light to impinge on the jaundiced neonate subject (S). As will be appreciated, the length and width of the flexible sheet 26a is designed in accordance with the expected size of the subject, e.g., neonate, toddler, adult, etc.

In the illustrated example, the flexible sheet 26b is functionally adapted as a swaddle wrap. For instance, the flexible sheet 26b is of suitable length to wrap entirely around the arms and torso of the subject (S). In this regard, the flexible sheet 26b may include a closure 32, such as but not limited to, a magnetic closure, hook and loop fastener, or snaps. In general, the flexible sheet 26b includes fewer LEDs 36 than the flexible sheet 26a. For example, on the side facing the subject (S) the flexible sheet 26b has a lit surface area of at least 5%.

The flexible sheet 26c is adapted as a body wrap for close proximity to the subject (S). For instance, the flexible sheet 26c is of suitable length to wrap entirely around the torso of the subject (S). In this regard, similar to the flexible sheet 26b, the flexible sheet 26c may also include a closure 32. Either of the flexible sheets 26b/26c may also be partially formed from materials that permits stretching, for enhanced fit of the flexible sheets 26b/26c on the subject (S). In general, the flexible sheet 26c includes fewer LEDs 36 than the flexible sheet 26a. For example, on the side facing the subject (S) the flexible sheet 26c has a lit surface area of at least 5% or at least 20%.

The controller 28 includes a control module 28a that serves to control operation of the light emitting diodes 36 of the flexible sheets 26. For example, the control module 28a includes hardware (e.g., one or more microprocessors), software, or both that is configured and/or programmed to perform the functions described herein. The hardware and software of the control module 28a, and thus the control functionality, are self-contained in the controller 28. However, in modified examples, portions of the hardware, software, or both may be remotely located and the phototherapy device 20 may, at least in part, be controlled remotely. In that regard, the control module 28a may also include one or more communication devices, such as but not limited to, a Universal Serial Bus port, an ethernet port, or a wireless device.

The controller 28 includes a user interface 28b, such as a keyboard, buttons, or a screen, through which a user can operate the phototherapy device 20. The controller 28 further includes a power source 28c. In the example shown, the power source 28c is a battery, although the power source 28c can additionally or alternatively include a power cord or adapter that permits the controller 28 to receive electrical power.

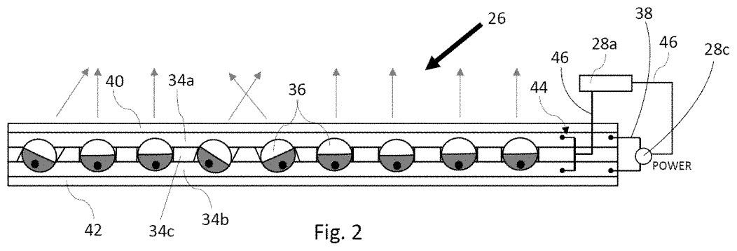

FIG. 2 illustrates a sectioned, representative view of the flexible sheet 26. In general, the flexible sheet 26 is relatively thin (e.g., less than 1 millimeter in thickness) and is highly flexible from use of thin polymer layers and avoidance of rigid soldering connections and avoidance of use of substrate-mounted LEDs. The flexible sheet 26 is formed of first and second electrical conduction layers 34a/34b and an electrical insulation layer 34c between the layers 34a/34b. The layers 34a/34b/34c may be bonded together by a bonding agent, such as an acrylic adhesive. The terminology “first” and “second” as used herein is to differentiate that there are two architecturally distinct components or features. It is to be further understood that the terms “first” and “second” are interchangeable in the embodiments herein in that a first component or feature could alternatively be termed as a second component or feature, and vice versa.

In this example, at least the first layer 34a is an optically transparent conductive polymer layer and the second layer 34b is metallic and/or optically transparent material. Conductive polymer layers may include, but are not limited to, conductive polymer films containing conductive metal oxides, carbon nanotubes, or other conductive allotropes of carbon. The metal of the layer 34b is not particularly limited and may be, but is not limited to, conductive metallic foil or conductive metallic paint. Likewise, the layer 34c is not particularly limited and may be, but is not limited to, polyethylene terephthalate, polyolefin, or polystyrene.

The LEDs 36 are disposed along the layer 34c, such as in voids in the layer 34c, between the electrical conduction layers 34a/34b. The layers 34a/34b connect in respective diode junctions to the LEDs 36 to form an electrical circuit 38 through which the LEDs 36 illuminate in response to an electrical input from the power source 28c. In additional examples, each LED 36 primarily emits light that has a wavelength from 400 nanometers to 550 nanometers. The LEDs 36 may be provided in one or more patterns, in a random two-dimensional array, or in a three-dimensional array by a stack of flexible sheets 26. In further examples, the flexible sheet 26 is provided, at least in part, as a micro-LED array, such as a printed LED available from NthDegree Technologies Worldwide Inc. or micro-LED from Rohinni LLC.

Depending on the implementation of the phototherapy device 20, the flexible sheet 26 may include one or more additional functional layers. For example, the flexible sheet 26 additionally includes an optically transparent polymer biocompatible layer 40 adjacent the first layer 34a (the side that faces toward the subject). For example, the polymer biocompatible layer 40 is polypropylene, polyester, combination thereof, or other material that is “inert” with respect to long-term exposure with human skin.

The flexible sheet 26 may further include a protective layer 42 adjacent the second layer 34b. In other words, the first and second layers 34a/34b are between the polymer biocompatible layer 40 and the protective layer 42. The protective layer 42 may serve to shield the LEDs 36 from moisture, mechanical damage, electrical shorting, or external substance that may hinder operation. For instance, the protective layer 42 is polypropylene, polyester, or a combination thereof.

The phototherapy device 20 additionally includes at least one meter 44 (FIG. 2) that is electrically coupled with the electrical circuit 38 and is operable to measure an electrical property of the electrical circuit 38. For example, the meter or meters 44 are selected from a resistance meter, Kelvin bridge meter, constant current meter, voltage meter, galvanometer, electrostatic meter, amperage meter, moving coil, moving magnet, hot wire, or integrating or virtual short. The electrical property or properties are selected from voltage, current, resistance, impedance, and combinations thereof.

The meter 44 can be located in the flexible sheet 26, in the controller 28, or in the flexible sheet 26 and the controller 28 (e.g., if there is more than one meter 44). The meter 44 is in communication with the control module 28a, which is also in communication with the power source 28c (connections represented at 46). For instance, an analog-to-digital converter may be used for communication between the meter 44 and the control module 28a. The control module 28a is configured to adjust the electrical input to the LEDs 36 responsive to the electrical property of the electrical circuit 38.

Performance of LEDs can vary over time and/or with temperature. To some degree the performance may be monitored by an external sensor, such as a temperature or photovoltaic sensor. The operation of the LEDs may then be modulated based on the feedback from the sensor. Under such an approach, however, there may be a delay between variations in LED performance, the sensing of the variations, and the response. Moreover, the external sensors measure locally, while conditions locally at the LEDs may differ (e.g., from body heat input), thereby leading to control actions that are inconsistent with more optimal performance of the LEDs.

In this regard, the phototherapy device 20 incorporates an assimilated control scheme in which, rather than reliance on external sensors for measuring external factors, the phototherapy device 20 internally measures the electrical circuit 38 directly with the meter 44 and then adjusts operation of the LEDs 36 based on the measurement.

In particular, the conductive polymer first layer 34a and the electrical properties of the diode junctions with the conductive polymer first layer 34a are sensitive to temperature fluctuations over the temperature range of interest (generally 20° C. to 42° C.) and thus cause variation in the performance of the LEDs 36. For example, the electrical impedance of the circuit 38 varies with temperature. The variation in the circuit 38, however, is somewhat counter-intuitive in that impedance of the circuit 38 varies inversely with temperature.

While performance variations are often a barrier to use, especially in applications where high performance is required (e.g., jaundice treatment), the phototherapy device 20 utilizes this sensitivity to facilitate enhancement of performance and reduction in variation. The electrical property of the circuit 38 is measured and then compared with pre-programmed relationships to temperature and/or light intensity (e.g., irradiance) of the LEDs 36. The control module 28a then adjusts operation of the LEDs 36 in order to regulate LED temperature and/or illumination.

As indicated above, the electrical property or properties are selected from voltage, current, resistance, impedance, and combinations thereof. From the electrical property the control module 28a can estimate the temperature and irradiance of the LEDs 36 in any single circuit 38 (i.e., a zone) by comparing an instant electrical property of the circuit 38 against reference electrical properties. The reference properties may be determined beforehand and programmed into the control module 28a by measuring the electrical properties at various control temperature conditions to establish temperature-property relationships. For instance, in a given one of the circuits 38, if the impedance decreases, the estimated temperature and irradiance has increased. In such a situation, the control module 28a will lower the voltage (electrical input), reducing the irradiance output and thereby lowering the temperature by reducing heat input. If the impedance increases, the estimated temperature and irradiance has decreased, meaning the irradiance is not be sufficient. In response the control module 28a increases the voltage, increasing current through the zone, increasing light output, and inputting heat into the device, raising the temperature.

In additional examples, the control scheme is based on one or more of a variable-voltage approach, a variable-current approach, or a variable-power approach. The following examples demonstrate aspects of each approach, in comparison to unregulated LEDs.

If the LEDs 36 were unregulated and under a fixed-voltage (e.g., −12V), a decrease in resistance across the circuit 38 from a temperature increase would lead to an increase in current draw from the power source 28c. The additional current draw would increase illumination and thereby increase heat generation from resistance across the LEDs 36. The heat would increase temperature and thereby further decrease resistance to generate more heat and a resulting increase in illumination. This process would repeat until a breakdown temperature is reached, e.g. by melting the polymer, and the LEDs 36 are no longer operable as desired.

Under a variable voltage approach (e.g., 3V to 60V), a meter 44 measures an instant resistance across the circuit 38. The instant resistance is compared to a control resistance for a target illumination output at a known ambient temperature. If the instant resistance differs, the control module 28a adjusts the voltage across the circuit 38 up or down, thereby matching the current through the circuit 38 to the control resistance.

Under a variable current approach (e.g., 0.1A to 10A), a meter 44 measures an instant voltage across the circuit 38. The instant voltage is compared to a control voltage for an intended illumination output at a known ambient temperature. If the instant voltage differs, the control module 28a adjusts the current across the circuit 38 up or down, thereby matching the voltage across the circuit 38 to the control voltage.

Under a variable power approach (e.g., 0.3W to 600W), a meter 44 measures amperage across the circuit 38. The instant amperage is compared to a control amperage for an intended LED temperature for the amperage consumption and ambient temperature. If the instant amperage differs, the control module 28a adjusts wattage up or down, to raise or lower the device temperature to maintain performance in the desired range, such as approximately 20° C. to 42° C. In further examples, the control module 28a utilizes two of the above approaches or all of the above approaches with multiple meters 44.

In further examples of the above approaches, the control module 28a includes one or more look-up tables, such as Table 1 below. For an instant electrical property measurement (N), the control module 28a compares to a control (reference) electrical property to determine a change or difference. The difference may represent an estimated LED temperature and/or LED illumination output. For given differences, such as a positive difference, no difference, or negative difference, there are corresponding control parameters. For instance, the control parameters may be to reduce the electrical property by a preset amount, increase the electrical property by a preset amount, or no change on the electrical property. For each given control parameter, there may also be corresponding communication messages, such as alarm, warning, operation OK, or no communication.

TABLE 1
Control Module 28a Look-up Table, Elec. Property

| Instant | Change in | Estimated | | |
|---|---|---|---|---|
| Elec. | Instant | Instant | | |
| Prop. | Elec. | Temp | Control | |
| Val. | Prop. Val. | (C.) | Parameter | Communication |
| N(lowest) | neg. | 40 | Turn Off | Alarm |
| Nᵢ₋₂ | neg. | 31 | X − 0.5 | Warning |
| Nᵢ₋₁ | neg. | 28 | X − 0.5 | None |
| Nᵢ | 0 | 25 | none | OK |
| Nᵢ₊₁ | pos. | 22 | X + 0.3 | None |
| Nᵢ₊₂ | pos. | 19 | X + 0.5 | Warning |
| N(highest) | pos. | 15 | Turn Off | Alarm |

In a further example, the control module 28a is configured to utilize one or more of the approaches above for multiple flexible sheets 26. For instance, the approach applied to each flexible sheet 26 is the same. In another example, the approach applied to each flexible sheet 26 is different and is customized or optimized for the function for which the flexible sheet 26 is adapted (whole body, body wrap, swaddle, etc.). In additional examples, the control module 28a utilizes multiple of the above approaches to control a single circuit 38 in order to regulate both illumination and temperature. For instance, the control module 28a is configured with an algorithm to process multiple electrical property inputs and output control parameters in response. In additional examples, the control module 28a is configured to regulate the individual flexible sheets 26 with respect to one another. For instance, the control module 28a is configured to shut-off one or more of the flexible sheets 26 in response to one of the flexible sheets 26 being disconnected from the control module 28a. Such an auto-shut-off may facilitate reduction in power consumption. In further examples, the auto-shut-off is alternatively or additionally triggered by one or more threshold irradiances. For instance, irradiance in one flexible sheet 26 that exceeds a threshold results in shut-off of all of the flexible sheets 26.

In additional examples, the irradiance and/or wavelength band is different between two or more of the flexible sheets 26. For instance, the irradiance is independently set by “zones” that correspond to different locations to be treated on the subject (S). As an example, each individual flexible sheet 26 is a zone, and/or an individual sheet 26 has multiple zones, wherein each zone is a separate electrical circuit 38 that is set and regulated with respect to a different irradiance that is desired for the particular location on the subject (S) which is to be treated by that zone. In this manner, the irradiance or other performance of the flexible sheets 26 can be set for more optimal treatment of particular locations on the subject (S). Different wavelength bands may be employed by using different diode types of LEDs 36 between the flexible sheets 26. Moreover, each flexible sheet 26 may itself have different diode types in order to provide different wavelength bands. The different diode types may be provided in different rows or arrays or along an edge of the flexible sheet 26. In a further example, the different diode types are provided between multiple flexible sheets that are stacked and formed of optically transparent materials such that light from lower layers travels through upper layers in the stack.

The zones of the phototherapy device 20 may also be adapted for differential phototherapy responses of the body. The body is a complex system of muscle, bones, fatty tissues, skin and other biologics, which may differ in a neonate as compared to an adult. For example, the layers of fat in a newborn may be thicker around the waist and buttocks, but thinner near the skull, feet, and palms. Additionally, a neonate undergoes a normal weight loss within the first week of life, complicating the application of phototherapy based on its initiation time after birth. A newborn 1 hour after birth will, therefore, have a different physiology than a newborn 5 days after birth. A comparison device that is not controlled as described herein utilizes only a monolithic light band, generally between 400 nanometers and 550 nanometers, which cannot be altered and is thus incapable of adapting to differential phototherapy responses of the body.

In further examples, the control module 28a also incorporates one or more types of external data, such as data on Total Serum Bilirubin (TSB) of a neonate subject (S) and/or external irradiance. Example control scheme look-up tables are shown below in Table 2 and Table 3. The TSB data is provided manually through the controller 28 and/or from a device that dynamically monitors TSB levels in the neonate, such as a trans-cutaneous skin color meter to estimate the TSB level. The irradiance data is provided from a photovoltaic meter or the like.

TABLE 2
Control Module 28a Look-up
Table, Total Serum Bilirubin (TSB)

| Instant | Change | |
|---|---|---|
| TSB | in TSB | |
| Prop. | Prop. | Communication |
| Val. | Val. | Protocol |
| N(lowest) | Decrease | Notify Lowest |
| Nᵢ₋₁ | Decrease | Notify Decrease |
| Nᵢ | Last Reading | Notify No |
| | | Change |
| Nᵢ₊₁ | Increase | Notify Increase |
| N(highest) | Increase | Alarm |

TABLE 3
Control Module Look-up Table, Irradiance

| | Change | | |
|---|---|---|---|
| | in | | |
| Instant | Instant | | |
| Irradiance | Irrad. | | |
| Prop. | Prop. | Control | |
| Val. | Val. | Parameter | Communication |
| N(lowest) | neg. | X − 0.5 | Alarm |
| Nᵢ₋₁ | neg. | X − 0.5 | None |
| N(set) | 0 | none | OK |
| Nᵢ₊₁ | pos. | X + 0.3 | None |
| N(highest) | pos. | X + 0.5 | Alarm |

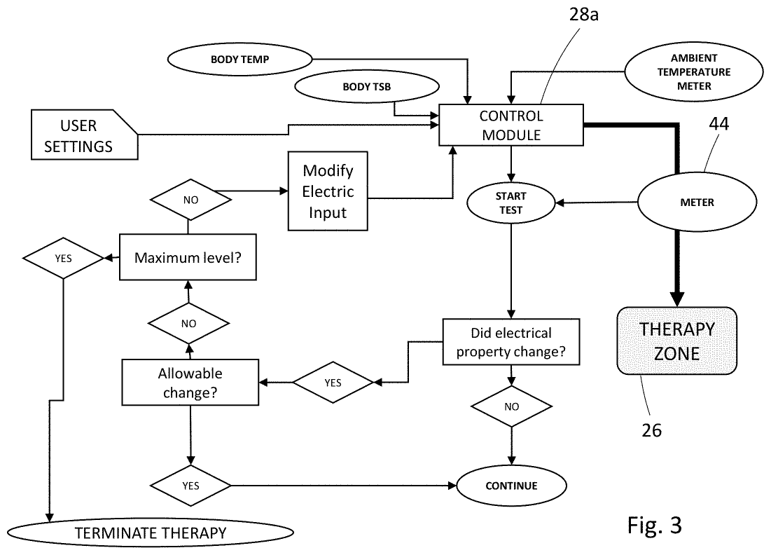

FIG. 3 shows an example flow diagram 50 of the control module 28a. At START TEST the control module polls the meter 44 for the instant electrical property. Next, the control module 28a compares the instant electrical property to the control electrical property to determine whether the instant electrical property has deviated. If there is no change, operation continues without any adjustment of electrical input into the flexible sheet 26. If there is change, the control module 28a determines whether the change is allowable, e.g., by comparison to a preset threshold. If the change is allowable, operation continues without any adjustment of the electrical input into the flexible sheet 26. If the change is not allowable, the control module 28a next determines whether the change exceeds a preset maximum level, which may indicate that LED temperature is above a desired limit or is trending to exceed a desired limit. If the change exceeds the preset maximum level, the control module 28a may terminate therapy by shutting off the LEDs 36. If the change does not exceed the preset maximum level, the control module 28a proceeds to modify the electrical input, e.g., in accordance with a look-up table as discussed above. As will be appreciated, the control module 28a may also receive external input data, such as TSB level, body temperature of the subject (S), and/or ambient temperature, in additional to user settings.

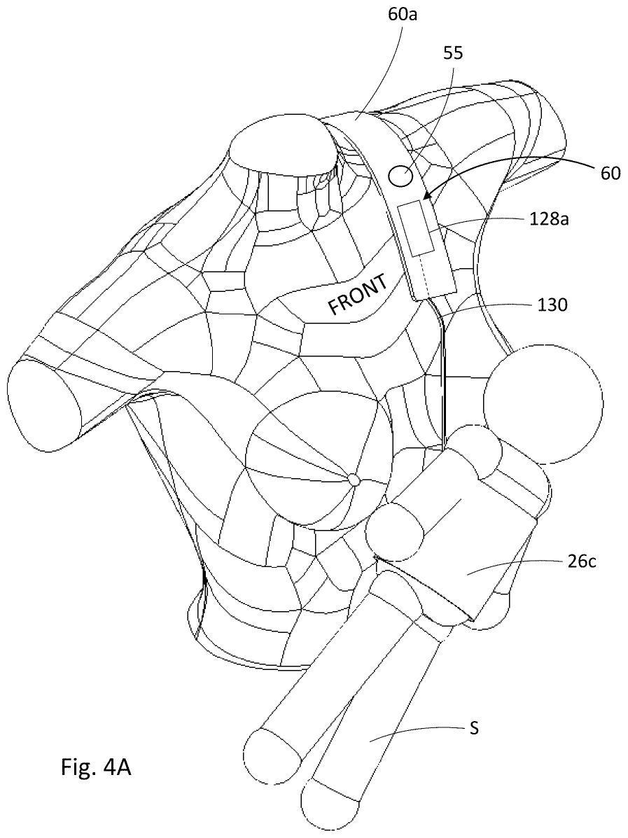

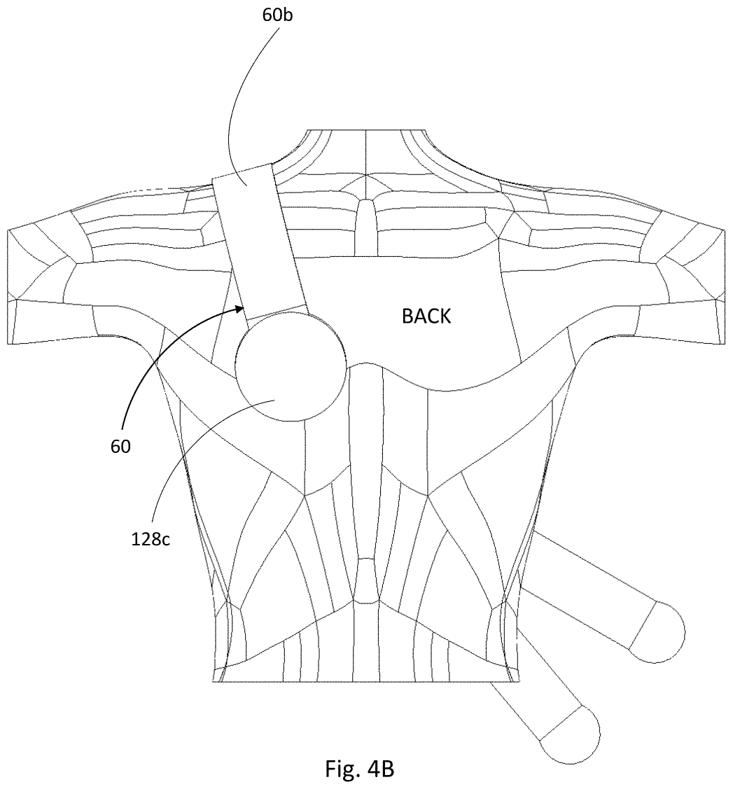

In further examples, the phototherapy device 20 is also reconfigurable for use in multiple different modes. The modes, or configurations, are functionally adapted to permit phototherapy under different situations. For example, one configuration is adapted for stationed use, such as in a crib or basinet, and another configuration is adapted for mobile or portable use, such as while breastfeeding. In these regards, the components of the phototherapy device 20 described above can also be considered to be part of a group of reconfigurable components, along with a wearable 60 shown in FIGS. 4A/4B.

The phototherapy system 20 is reconfigurable between first and second configurations. The first configuration is represented in FIGS. 1A/1B, in which the flexible sheets 26a/26b/26c are connected via the network of electrical connections 30 to a first control module, i.e., the control module 28a, which operates as discussed above. In this configuration, the wearable 60 is not used.

The second configuration is represented in FIGS. 4A/4B, while in use on a mother for breastfeeding. The wearable 60 is configured to be attached on anatomy of a human body. For example, the wearable 60 includes a shoulder wrap 60a that incorporates a shepherd's hook 60b that is shaped to fit over the shoulder of a wearer. A power source 128c (e.g., battery) is incorporated into the back of the wearable 60 in this example, and the front includes a second control module 128a. The power source 128a in the back facilitates a weight balance to facilitate fit and comfort. The power source 128c and the control module 128a are electrically connected with the flexible sheet 26c via body electrical connection 130.

In this second configuration, the flexible sheet 26c is disconnected from the network of electrical connections 30 and the first control module 28a, and is connected to the second control module 128a via a body electrical connection 130. The second control module 128a is configured to control the flexible sheet 26a to adjust electrical input to the flexible sheet 26a, as described above for the control module 28a and incorporated herein.

The flexible sheet 26c (e.g., a body wrap in this example) is thus adapted to be used in two different configurations. In the first configuration, the flexible sheet 26a facilitates phototherapy while stationed, such as in the basinet 24. In that regard, a portion of the flexible sheet 26c (and also of flexible sheet 26b) may be optically transparent in order to permit light from the flexible sheet 26a to reach the subject (S). The flexible sheet 26c can be removed, such as by disconnecting the flexible sheet 26c from the electrical connections 30, and then used with the wearable 60 by connecting the body electrical connection 130. As an example, a neonate subject (S) may undergo phototherapy in the basinet 24 for a period of time. Upon feeding time, the flexible sheet 26c is disconnected and the neonate subject (S) is removed from the basinet 24 for feeding. The flexible sheet 26c is then connected to the wearable 60 such that the phototherapy continues while feeding, without necessarily having to remove the flexible sheet 26c from the neonate subject(S). Once finished, the flexible sheet 26c can be disconnected from the wearable 60 and then reconnected to the controller 28 at the basinet, where the phototherapy can be further continued. As will be appreciated from the present disclosure, the phototherapy device 20 is not necessarily limited to jaundice treatment of neonates and may be adapted for other treatments, such as but not limited to, as an anti-pathogen treatment for humans or animals.

Although a combination of features is shown in the illustrated examples, not all of them need to be combined to realize the benefits of various embodiments of this disclosure. In other words, a system designed according to an embodiment of this disclosure will not necessarily include all of the features shown in any one of the Figures or all of the portions schematically shown in the Figures. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

What is claimed is:

1. A phototherapy device comprising:
at least one flexible sheet including
first and second electrical conduction layers, the first electrical conduction layer including a conductive polymer,
an electrical insulation layer between the first and second electrical conduction layers, and
light emitting diodes (LEDs) disposed along the electrical insulation layer between the first and second electrical conduction layers, the first and second electrical conduction layers and the LEDs being part of an electrical circuit in which the LEDs illuminate responsive to an electrical input;
at least one meter to measure an electrical property of the electrical circuit; and
a control module in communication with the at least one meter and configured to adjust the electrical input responsive to the electrical property of the electrical circuit.

2. The phototherapy device as recited in claim 1, wherein the electrical property is selected from the group consisting of voltage, current, resistance, impedance, and combinations thereof.

3. The phototherapy device as recited in claim 1, wherein the first electrical conduction layer is an optically transparent conductive polymer layer, and the second electrical conduction layer is metallic.

4. The phototherapy device as recited in claim 3, further comprising a polymer biocompatible layer adjacent the first electrical conduction layer, the polymer biocompatible layer selected from the group consisting of polypropylene, polyester, and combinations thereof.

5. The phototherapy device as recited in claim 4, further comprising a protective layer adjacent the second electrical conduction layer, the first and second electrical conduction layers being between the polymer biocompatible layer and the protective layer.

6. The phototherapy device as recited in claim 3, including a plurality of electrical connections, the at least one flexible sheet includes first and second flexible sheets that are connectable with the control module via the electrical connections, and the control module is configured to adjust the electrical inputs of the first and second flexible sheets independently of each other.

7. The phototherapy device as recited in claim 6, further comprising a shoulder wrap that has a battery and a body electrical connection, wherein the first flexible sheet is disconnectable from the electrical connection to the control module and is connectable with the shoulder wrap via the body electrical connection for portable use of the first flexible sheet.

8. The phototherapy device as recited in claim 7, wherein the shoulder wrap includes a shoulder wrap control module, and the shoulder wrap control module is also configured to adjust the electrical input responsive to the electrical property of the electrical circuit.

9. The phototherapy device as recited in claim 7, wherein the shoulder wrap includes a transcutaneous bilirubin monitor.

10. The phototherapy device as recited in claim 7, wherein the control module deactivates the second flexible sheet responsive to disconnection of the first flexible sheet from the frame.

11. The phototherapy device as recited in claim 1, wherein the at least one meter includes at least two different meters selected from the group consisting of a voltage meter, an amperage meter, and a resistance meter.

12. The phototherapy device as recited in claim 1, wherein the electrical property is resistance or impedance, and responsive to a decrease in the resistance or impedance the control module is configured to reduce a temperature of the LEDs by changing the electrical input to increase the resistance or impedance.

13. The phototherapy device as recited in claim 1, wherein the control module is configured to estimate a temperature of the LEDs based on the electrical property of the electrical circuit and maintain the temperature below 42° C. by adjusting the electrical input.

14. The phototherapy device as recited in claim 1, wherein at least a portion of the at least one flexible sheet is optically transparent.

15. The phototherapy device as recited in claim 1, wherein the at least one flexible sheet is less than 1 millimeter thick, each of the LEDs has a cross-sectional area taken parallel to the at least one flexible sheet when flat of 0.006 square millimeters to 0.275 square millimeters, and the LEDs primarily emit light that has a wavelength from 400 nanometers to 550 nanometers.

16. A method of treating jaundice using the phototherapy device as recited in claim 15, the method including moving the at least one flexible sheet to be in proximity of a jaundiced neonate and causing the light to impinge on the jaundice neonate.

17. The phototherapy device as recited in claim 1, further comprising a mattress, a bassinet, or both, situated adjacent the at least one flexible sheet.

18. A phototherapy device comprising:
a reconfigurable group of components including:
first and second flexible sheets each having light emitting diodes (LEDs),
a first control module;
a network of electrical connections;
a wearable that is configured to be attached on anatomy of a human body, the wearable including a second control module, a battery, and body electrical connection;
the reconfigurable group of components being operable in the following configurations:
a first configuration in which the first and second flexible sheets are connected via the network of electrical connections to the first control module, and the first control module is configured to adjust electrical inputs to the first and second flexible sheets, and
a second configuration in which the first flexible sheet is disconnected from the network of electrical connections and first control module and is connected to the body electrical connection, and the second control module is configured to adjust electrical input to the first flexible sheet.

19. The phototherapy device as recited in claim 17 wherein the LEDs are part of an electrical circuit in which the LEDs illuminate responsive to an electrical input, further comprising a first meter to measure an electrical property of the electrical circuit, and the first control module is configured to adjust the electrical input responsive to the electrical property of the electrical circuit.

20. The phototherapy device as recited in claim 18, further comprising an additional meter to measure the electrical property of the electrical circuit, and the second control module is configured to adjust the electrical input responsive to the electrical property of the electrical circuit.

21. The phototherapy device as recited in claim 17, wherein the first and second flexible sheets each include:
first and second electrical conduction layers, the first electrical conduction layer including a conductive polymer,
an electrical insulation layer between the first and second electrical conduction layers, and
the LEDs are disposed along the electrical insulation layer between the first and second electrical conduction layers, the first and second electrical conduction layers and the LEDs being part of an electrical circuit in which the LEDs illuminate responsive to an electrical input.
